(19) United States
(12) Patent Application Publication
(10) Pub. No.: US 2025/0303030 A1
(43) Pub. Date: Oct. 2, 2025

(54) LOW PROFILE HYDROGEL-BASED ORTHOPEDIC IMPLANTS

(71) Applicant: Sparta Biomedical, Inc., Madison, NJ (US)

(72) Inventors: Benjamin J. WILEY, Durham, NC (US); Demetrius SIACHAMES, Madison, NJ (US); Spencer WHITE, Madison, NJ (US); Audrey POLITOWICZ, Madison, NJ (US)

(21) Appl. No.: 19/096,647

(22) Filed: Mar. 31, 2025

Related U.S. Application Data
(60) Provisional application No. 63/572,170, filed on Mar. 29, 2024.

(51) Int. Cl. A61L 31/14; A61B 17/00; A61B 17/84; A61L 31/12

(52) U.S. Cl. CPC A61L 31/145; A61B 17/84; A61L 31/129; A61B 2017/00526; A61L 2400/18; A61L 2430/06; A61L 2430/24

(57) ABSTRACT

Methods, hydrogel compositions, and apparatuses (e.g., implants) that address the need for preventing subsidence of implants through cortical bone layers and exposure of hydrogel compositions to bone and other degradatory interfaces, by securing hydrogel compositions to and within implant structures via a crimping process.

### CLAIM OF PRIORITY

This patent application claims priority to U.S. Provisional Patent Application No. 63/572,170, titled “LOW PROFILE HYDROGEL-BASED ORTHOPEDIC IMPLANTS” filed on Mar. 29, 2024, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Human cartilage is a semipermeable, avascular tissue that receives nutrients via diffusion from synovial fluid during motion of the joint. Synovial fluid is a viscous fluid that surrounds articulating joints. Due to these unique properties, cartilage has viscoelastic and lubricating properties. The main function of cartilage is to provide a smooth surface for joint articulation and facilitate the transmission of loads with a low frictional coefficient. Water is the most abundant component of cartilage, which allows it to provide a lubricating surface in joints while also withstanding significant loads. Replicating these physical and mechanical properties of natural cartilage is essential for any artificial cartilage replacements. Due to the challenges that this presents, few cartilage replacements are readily available.

Historically the only choices available to patients with cartilage damage, especially the cartilage of an articulating joint, such as a knee or elbow, were to initially do nothing if the extent of the damage was only relatively minor in scope, which sooner or later usually led to a worsening of the condition and further damage to the cartilage and to the joint itself, with the patient feeling discomfort and pain when using the joint, thus ultimately requiring a complete joint replacement to restore mobility; or, if the extent of the damage was significant to start with, to immediately perform a complete joint replacement.

When a patient suffers from the loss or damage of cartilage, the most common treatments are matrix-induced autologous chondrocyte implantations (MACI), osteochondral autografts, and total knee replacements. However, each of these treatment options present high costs and risks. MACI can only be used for patients that are young and have good regenerative abilities, requires two operational procedures, and risks cartilage overgrowth. A few downsides of osteochondral autografts are that it consists of an invasive surgical procedure, and it requires donor tissue, which is incredibly limited and risks rejection by the body. A total knee replacement is a heavily invasive and expensive surgical procedure that is followed by a relatively long recovery and potential surgical complications. Many replacement knee, elbow joints and shoulder joints typically have had a maximum active useful life of only about ten years, due to wear and tear and erosion of the articulating surfaces of the joint with repetitive use over time, thereby necessitating periodic invasive surgery to replace the entire joint. It would be useful to provide implants having much longer useful lives.

In addition, joint replacements are problematic in young patients where their skeletal bone structure is not fully developed, causing the artificial joint to be outgrown, presenting the potential prospect of future surgeries to continue replacing the outgrown joint. For a very young patient this meant that they would have to face the prospect for several more such surgeries over their lifetime, notwithstanding progress and improvements in the wearability of materials used for joint surfaces that have been made and continue to be made as new materials are developed.

A common diagnosis that causes a patient to need a total knee replacement is osteoarthritis (OA), a degenerative joint disease in which tissues in the joint break down over time. This disease affects approximately 3.6% of the population globally, causing moderate to severe disability in 43 million people, making it the 11th most debilitating disease worldwide. OA begins with damage to the cartilage between two bones in a joint. This damage consists of surface fibrillation, irregularity, and focal erosions. If this initial breakdown in cartilage is not addressed, the OA will continue to develop, causing damage to tendons and bone and eventual loss of function. It would be beneficial to provide implants that avoid the pitfalls mentioned above; it would also be helpful to provide implants that may remain securely contained within the cortical bone layer to prevent subsidence of implants beyond the cortical bone layer and avoid cyst formation. It would also be useful to provide apparatuses and methods for protecting the bone/implant interfaces from exposure to potentially degrading elements.

### SUMMARY OF THE DISCLOSURE

This disclosure relates generally to variations of artificial cartilage materials in implants suitable for repair of cartilage, including hydrogel composites and methods and for attaching a hydrogel composite including a compressed sheet of bacterial cellulose (BC) impregnated with a hydrogel to a surface of an implant. Described herein are methods, hydrogel compositions, and apparatuses (e.g., implants) that address the need for preventing subsidence of implants through cortical bone layers and exposure of hydrogel compositions to bone and other degradatory interfaces, by securing hydrogel compositions to and within implant structures via a crimping process.

Also described herein are procedures, hydrogel compositions, and apparatuses (e.g., devices and systems, including implants), and method of making and using such apparatuses, that are designed for the replacement of damaged cartilage in the femoral condyle. In some examples, an implant consists of a hydrogel composition secured to a titanium base. The characteristics of one design may allow for the implant to replace focal erosions in the knee without causing the formation of cysts or bone degeneration.

These methods and apparatuses may be used to treat disorders, including but not limited to osteoarthritis (OA) with less costs and risks than the existing treatments. Any of these methods and apparatuses may include the use of a hydrogel, and in particular a hydrogel impregnated into a bacterial cellulose (BC) matrix. Hydrogels are smooth, elastic biomaterials that exhibit high water content. These three-dimensional polymer networks can absorb large amounts of water while retaining structure, yielding high mechanical strength and cartilage-like characteristics. The hydrogel-based apparatuses described herein may provide a biocompatible treatment for disorder such as OA that replace damaged cartilage before the occurrence of bone damage or following repair of such bone damage.

The methods and apparatuses may also avoid problems associated with other proposed apparatuses, including susceptibility to subsidence or reduction of the height of the implant if an implant penetrates the compact or cortical bone layer (the outermost layer of bone) which surrounds the diaphysis and metaphysis of long bones. This may affect the performance and lifetime of the implant.

The bacterial cellulose-hydrogels (BC-hydrogels) described herein include polymer networks that may be swollen with water and used as part of an implant for replacement of cartilage because these BC-hydrogels can have similar mechanical and tribological properties as natural cartilage. Such hydrogels may exhibit superior wear characteristics because their surfaces principally consist of water, which serves to lubricate the surface and lower the coefficient of friction. Such superior wear characteristics may result in a longer-lasting joint replacement. Although traditional hydrogels may be difficult to use, and in particular have been difficult to integrate with bone on their own, since they lack the appropriate porosity and surface chemistry, the methods and apparatuses described herein may resolve these difficulties. Hydrogels have traditionally been difficult to attach to materials, such as titanium, that integrate with bone. Previous work required the use of cements or through a clamp. However, cements represent an extra cost and may introduce additional toxic compounds that may be deleterious to their use in an implant that is meant to reside in the body for decades. Clamps are also limited in that they may be able to attach hydrogels to an implant with a limited set of geometries, such as circular or cylindrical geometries, that enable an even pressure to be applied around the clamp radius. Clamps are also limited in that they can only be applied to relatively stiff materials that resist the clamp force. If a clamp is applied to a soft material, it will simply deform, and the material will not be tightly attached to the surface. The methods and apparatuses described herein enable the attachment of hydrogels to titanium or other appropriate material implants without the use of a clamp or cement, especially to complex geometries that mimic the human joint anatomy.

For example, an orthopedic implant may include: a head having an upper surface, an undercut region, and a side region extending between the upper surface and the undercut region; a bacterial cellulose (BC) layer impregnated with a hydrogel covering the upper surface, the side region and at least partially over the undercut region; a retaining plate secured against the undercut region so that the BC sheet of BC impregnated with the hydrogel is clamped between the retaining plate and a surface of the undercut region; and a post extending from the head and through the retaining plate.

In any of these implants, a portion of the BC sheet impregnated with hydrogel on the undercut region may form a plurality of channels in the BC sheet extending radially inward from the edge region.

The orthopedic implant may further include a retaining ring secured to the post and holding the retaining plate in position.

In any of these implants, the side region may not be covered by the retaining plate.

In any of these implants, the retaining ring may be crimped around the post.

In any of these implants, the retaining ring may be crimped into a recess on the post.

In any of these implants, the BC sheet may be impregnated with hydrogel is annealed.

In any of these implants, the BC sheet impregnated with hydrogel may be impregnated with a polyvinyl alcohol (PVA) hydrogel.

In any of these implants, the retaining plate may include a disk.

In any of these implants, the upper surface of the head of the implant may be contoured by molding to have a smooth external surface.

In any of these implants, the post may include a plurality of engaging edges along the length, configured to engage the bone.

When used for partial knee resurfacing, the implant may be configured to wear an opposing cartilage surface to an extent not significantly greater than the extent to which cartilage wears cartilage. A top bearing surface of the implant may have a coefficient of friction (COF) that is not statistically different from that of cartilage.

The implants described herein may be configured as a medical implant, and may include a tissue engaging portion (e.g., a bone engaging portion such as a rod, screen, nail, etc.).

The nanofiber network may be secured to the implant (e.g., to a porous surface of the implant) by any appropriate method.

The implant may be formed of any appropriate biocompatible material. For example, the surface of the implant body may be titanium. The surface of the implant body may be one or more of: a stainless steel alloy, a titanium alloy, a Co—Cr alloy, tantalum, gold, niobium, bone, Al oxide, Zr oxide, hydroxyapatite, Tricalcium phosphate, calcium sodium phosphosilicate, poly(methyl methacrylate), polyether ether ketone, polyethylene, polyamide, polyurethane, or polytetrafluoroethylene.

In general, the nanofiber network may be coupled to the top bearing surface of the implant. The cross-linked cellulose nanofiber network may be attached over the top load surface by clamping. For example, the nanofiber network may be bonded by cement to the top load surface; in some examples, the cement is not bonded to the hydrogel; the cement is only bonded to the nanofiber network. Alternatively, in some examples the nanofiber networks may be coupled to the implant, so that the nanofiber network, is secured over the top bearing surface without the use of a chemical adhesive, such as an epoxy. Instead, the nanofiber network may be secured over the top bearing surface by a clamp. For example, a clamp may secure the nanofiber network (e.g., one or more sheets of BC) over the top bearing surface around a periphery of the top bearing surface. Thus, in general, the use of an adhesive (such an epoxy) is optional.

Any appropriate implant may be used. The surface of the implant (e.g., top bearing surface, which may be equivalently referred to as simply the bearing surface) may be at least at the region to which the nanofiber network is attached over, may be titanium, stainless steel, etc. the bearing surface (e.g., top bearing surface) may be convex, flat, concave, or some mixture of these. For example, the surface of the implant body may comprise one or more of: a stainless steel alloy, a titanium alloy, a Co—Cr alloy, tantalum, gold, niobium, bone, Al oxide, Zr oxide, hydroxyapatite, Tricalcium phosphate, calcium sodium phosphosilicate, poly(methyl methacrylate), polyether ether ketone, polyethylene, polyamide, polyurethane, or polytetrafluoroethylene.

Also described herein are methods of making and/or using these implants. For example, described herein are implants, comprising: an outer surface; and a bacterial cellulose (BC) layer (e.g., a compressed BC layer) impregnated with a hydrogel covering the outer surface, wherein the BC impregnated with hydrogel has a thickness of between about 0.5-8 mm, and wherein a cellulose fiber density of the BC is between about 0.001 g/mm³ to 0.00001 g/mm³.

In some examples, the BC layer impregnated with the hydrogel of the implants comprises a plurality of layers of compressed BC layers impregnated with the hydrogel.

In some examples, the outer surface of the implants comprises a partially spherical shape. In some examples, the implant surface is concave or convex.

In some examples, the BC layer impregnated with a hydrogel is configured to have a water content of between 30%-70% when hydrated.

In some examples, the hydrogel comprises a polyvinyl alcohol (PVA) hydrogel.

In some examples, the implant is a titanium implant.

In general, the methods and apparatuses described herein may be used with any of the methods, apparatuses and compositions described in International Patent Application No. PCT/US2021/040031, titled “NANOFIBER REINFORCEMENT OF ATTACHED HYDROGELS,” filed on Jul. 1, 2021, which is herein incorporated by reference in its entirety.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings.

FIG. 1A shows an exemplary process flow for orthopedic implants to form a femoral head component of a hip joint without the use of a clamp or cement.

FIG. 1B shows an example of a method of forming an implant as described herein.

FIG. 1C shows an exemplary layer of a laser-cut sheet of bacterial cellulose (BC) having a floral shaped pattern.

FIGS. 1D-1E show an example of steps for securing a BC-hydrogel to the head of an implant.

FIG. 1F shows an example of additional steps of a method for forming an implant having a BC-hydrogel.

FIG. 1G shows an example of steps of a method for forming an implant having a BC-hydrogel including infiltrating the BC with the hydrogel.

FIGS. 1H-1I show examples of an implant during the fabrication process.

FIG. 1J shows an example of an implant before molding.

FIGS. 1K-1L show examples of an implant after molding.

FIG. 1M is an example of a disassembled two-part mold fixture.

FIG. 1N is an example of an assembled two-part mold fixture.

FIG. 1O schematically illustrates an example of a molding process that may be used for forming an implant having a BC-hydrogel.

FIGS. 1P-1R show examples of an implant having a BC-hydrogel.

FIG. 2A shows an example of an implant having an outer surface with a hydrogel impregnated bacterial cellulose (BC).

FIG. 2B shows the implant of FIG. 2A after formation of a crimp to better retain the hydrogel impregnated BC.

FIG. 3 shows an axial view of the implant with a retaining plate temporarily securing the portion of the sheet of BC covering the undercut region of the head.

FIG. 4 shows a bottom view of the implant including channels (spaces) between the folded-over BC; these channels may allow or enhance at least partial polyvinyl alcohol (PVA) penetration in this portion of the implant.

FIG. 5 is a side perspective axial view of an implant with a metal backplate.

FIG. 6 schematically illustrates one method of forming an implant as described herein.

FIG. 7 schematically illustrates a method of forming an implant as described herein.

FIG. 8 is a chart illustrating a method of implanting an implant.

FIGS. 9A-9B illustrate an example of components of an implant as described herein. FIG. 9A shows a section through the metal core of the implant. FIG. 9B shows an exploded view of the metal core, as well as a crimping ring and retaining plate.

FIGS. 10A-10B illustrate an assembled implant similar to that of FIGS. 9A-9B with the BC-hydrogel included. FIG. 10A sows a section through an implant. FIG. 10B shows a side view of the implant of FIG. 10A.

FIGS. 11A-11B schematically illustrate a method of forming an implant as described herein.

### DETAILED DESCRIPTION

Described herein are methods and apparatuses (e.g., systems and devices, including implants) that may provide medical implants, and in particular medical implants having a head region, such as but not limited to, screws, pins, anchors, etc., having a robust and more easily fabricated outer hydrogel covering. In some cases, these implants may resist subsidence through a cortical bone layer into which they are inserted, and may limit exposure of the hydrogel material to bone and other depredatory interfaces, by securing hydrogel compositions to and within implant structures via coupling to an undercut region and/or by using a crimping process.

Although the methods and apparatuses described herein are primary described in the context of bacterial cellulose (BC) and polyvinyl alcohol (PVA), e.g., BC impregnated with PVA (also referred to herein as BC-PVA), other hydrogels may be used in addition to or instead of PVA. For example, these methods and apparatuses may be used with hydrogels comprising a bacterial cellulose (BC) network infused/impregnated with both polyvinyl alcohol (PVA) and poly(2-acrylamido-2-methyl-1-propanesulfonic acid sodium salt (PAMPS), referred to as BC-PVA-PAMPS hydrogels, as described in International Patent Application No. PCT/US2021/040031, which is incorporated herein by reference in its entirety.

The methods and apparatuses described herein include may generally allow secure attachment of the hydrogel (e.g., a BC-PVA hydrogel) to conform to surface of virtually any shape, including concave, convex, saddle, conical, etc., and including abutments of surfaces, including curved surfaces, such as the surface geometries in human joints. Thus, the methods and apparatuses described herein may include a total or partial joint implant with a hydrogel that covers the bearing surface, i.e., the surface that is in contact with the opposing joint surface. The hydrogel may be a BC-PVA hydrogel, or any other BC-based hydrogel, and does not require a clamp for fixation.

In some cases the bacterial cellulose (BC) material forming part of the hydrogel described herein may be formed by fusing multiple pieces or layers (including layer of the same piece) of BC together, with a range of tensile strengths as described herein. Fusion of difference regions/layers of BC as described herein may allow the creation of curved BC surfaces that are smooth and/or that lack cracks that may otherwise cause that surface to tear. The resulting BC surface may be infused/impregnated with hydrogel (e.g., PVA).

In some examples, the methods and apparatuses described herein may avoid the use of a clamp or other securement feature when securing the hydrogel (e.g., the BC-PVA) to the surface. In particular, these methods may avoid the use of a clamp around an outer perimeter of the head of the implant. In some cases, these methods and apparatuses may include molding the hydrogel around an object with undercuts or other features such that the geometry of the BC material (hydrogel) and the implant itself may ensure that the hydrogel remains fixed to the surface of the implant with the strength of the hydrogel. Additionally or alternatively, a securing material (e.g., a retaining plate) may be secured against a portion of the BC-PVA either during the fabrication (e.g., as a temporary securement) and/or after fabrication (e.g., after impregnating the BC with the hydrogel), and in particular, securing the undercut region.

In general, these methods and apparatuses may provide a consistent thickness of the hydrogel material. For example, the methods described herein may include the use of a vacuum to enable the formation of a hydrogel with a consistent thickness across the surface of the implant, thereby ensuring that the hydrogel thickness remains consistent (e.g., approximately the same) across a curved surface. For example, these methods may provide a consistency of thickness over even irregularly-shaped, and/or curved, surfaces that is, e.g., within +/−10% of the thickness of the surface (e.g., within +/−7%, within +/−5%, within +/−3%, within +/−1% e.g., within about +/−0.2 mm, for example).

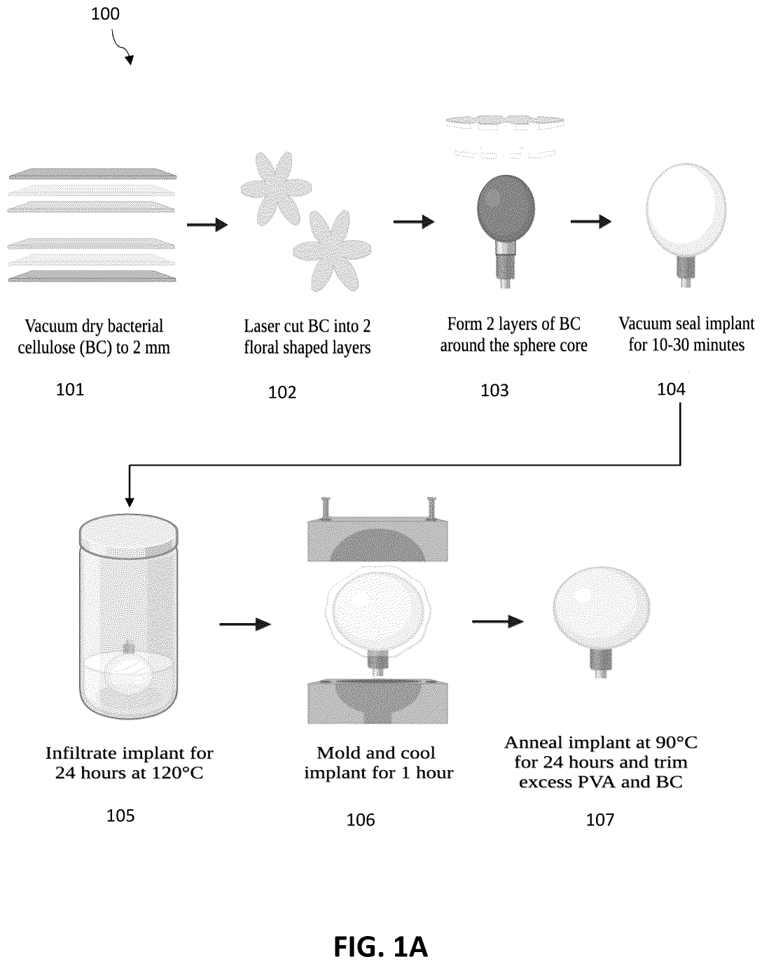

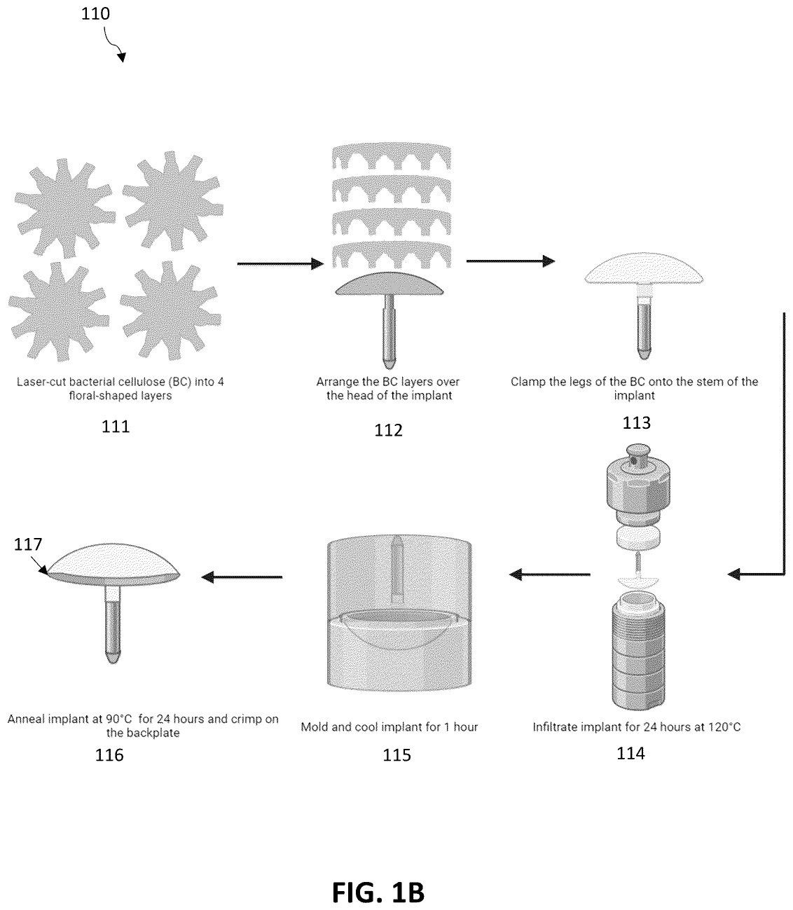

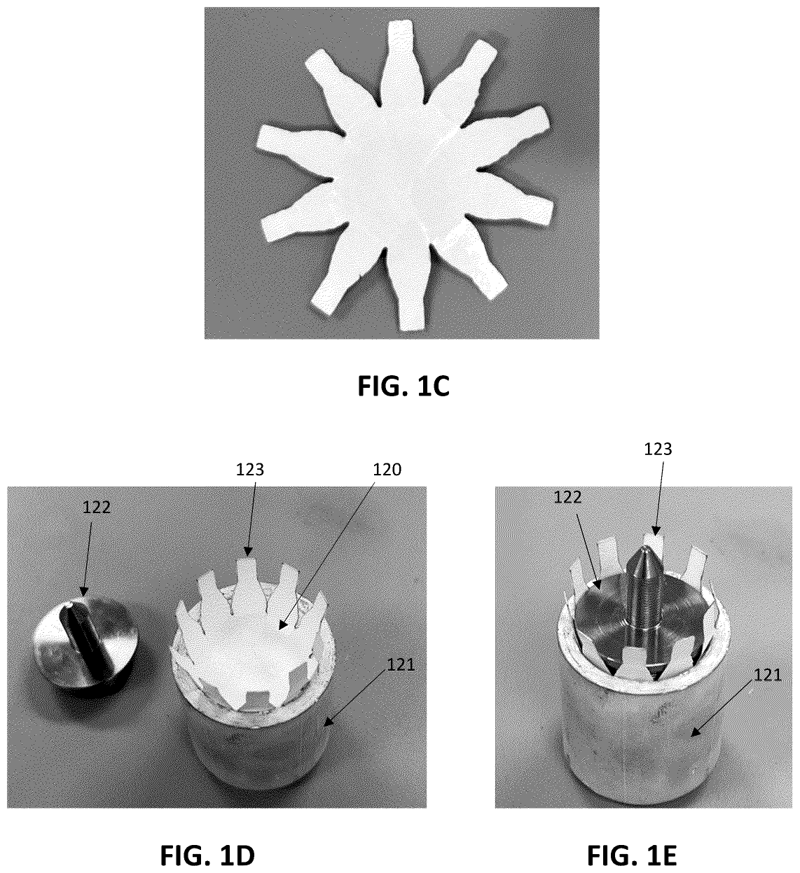

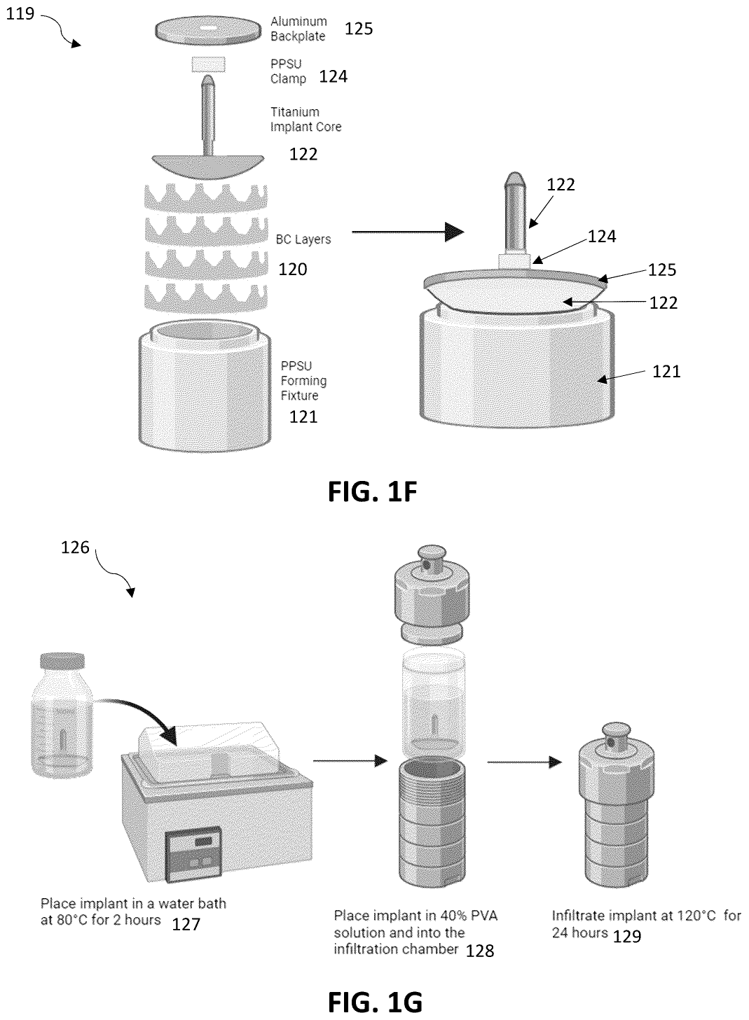

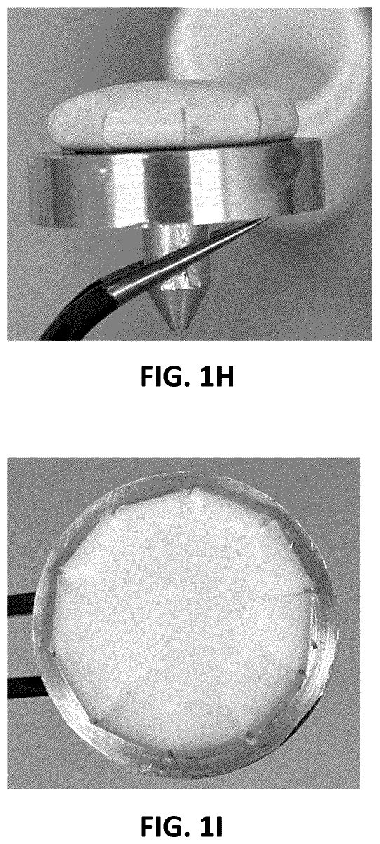

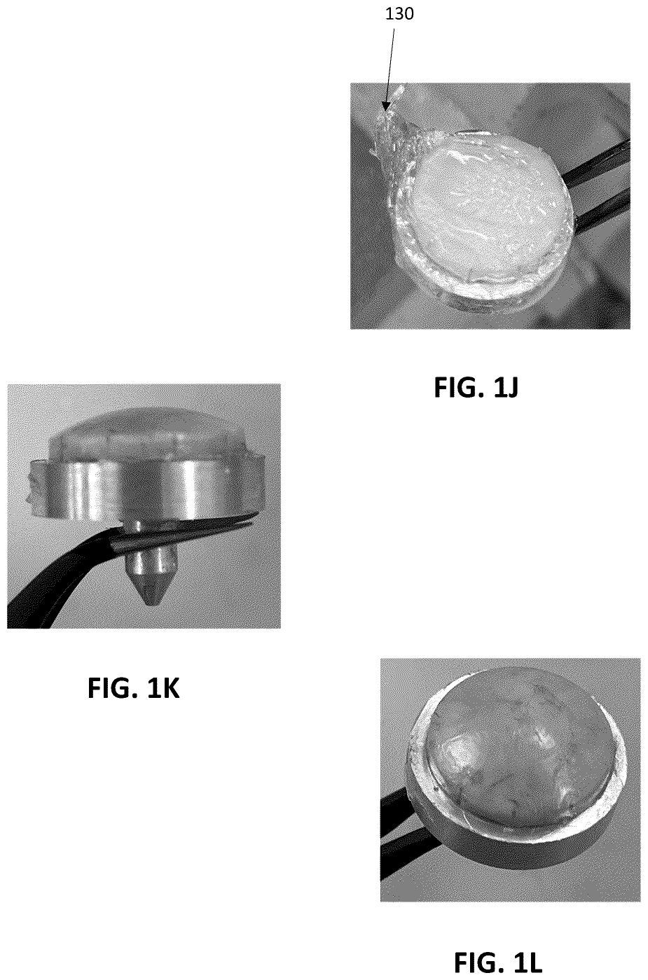

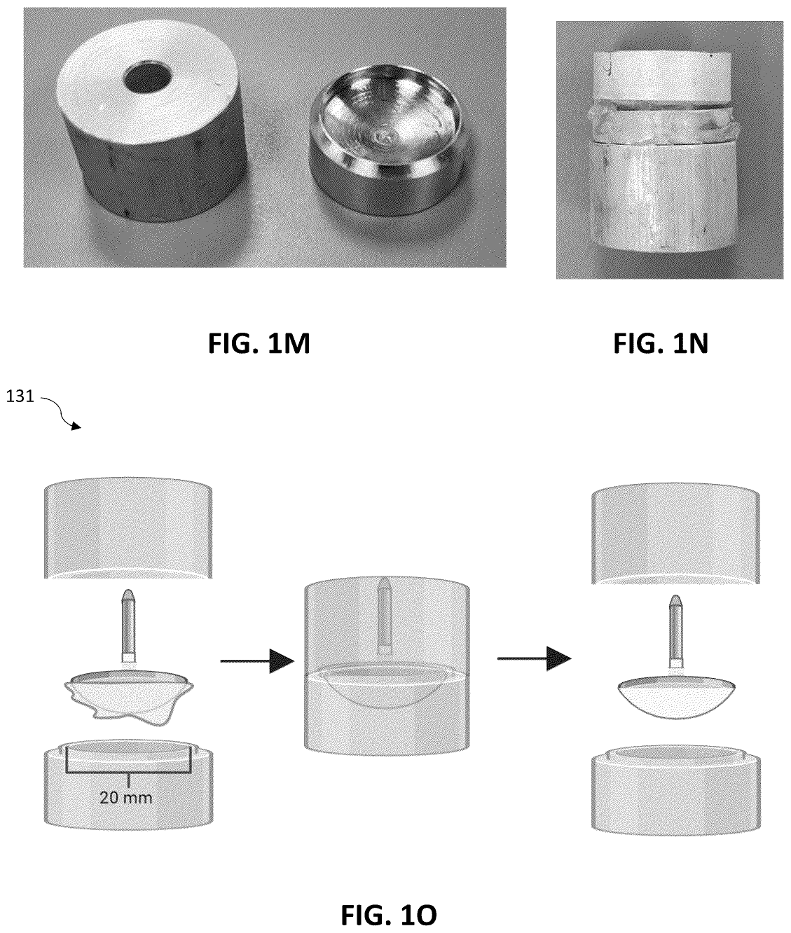

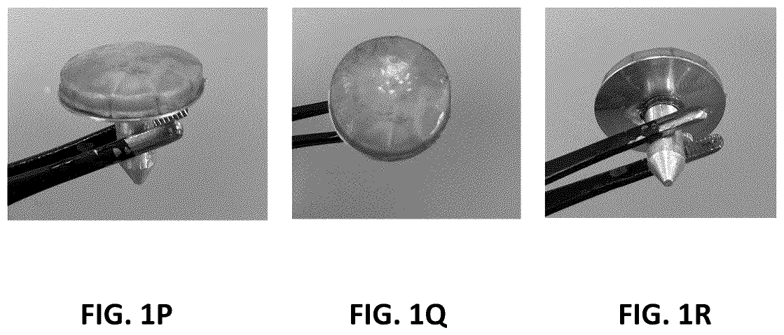

As mentioned above, the methods described herein allow attachment of a hydrogel, including a BC-PVA hydrogel, to an arbitrarily-shaped surface. For example, FIG. 1A shows an exemplary process flow 100 for orthopedic implants to form a femoral head component of a hip joint without the use of a clamp or cement including compression step 101, laser cutting step 102, layering BC on sphere surface step 103, compression sealing the implant step 104, infiltrate step 105, molding and cooling step 106, and annealing and trimming step 107. FIG. 1A illustrates an example of a process for attaching a hydrogel to a geometry that mimics human anatomy, in this case, represented by a sphere that mimics the geometry of the femoral head.

As shown in FIG. 1A, the process 100 for forming the femoral head of the hip joint utilizes a sphere-shaped implant surface such as a metal core and two layers of floral-shaped BC. This process, however, can be adapted to a variety of different joints dependent on the implant surface design and appropriate geometric cutting of BC. Within the human body, the shoulder joint is similar to the ball-and-socket hip joint, indicating that a similar process can be configured to form an implant that mimics the human humeral head. These implant surfaces can also be designed as concave cavities to mimic the acetabulum and glenoid fossa, the socket regions of the hip and shoulder joints. The implant surfaces may also be designed to mimic the triangular shape of the knee joint and elbow joints. The concave cavities may have small keys or slots 201 on their surface to allow for the BC and PVA to sit and adhere to the surface when vacuumed. According to other examples, a flat or other shaped surface such as a convex surface may be used instead of a metal core. The implant surfaces may contain both convex and concave surfaces depending on a desired joint, or according to yet other examples, the implant surface may be completely or partially flat.

To form an orthopedic implant, one sheet (20 cm×10 cm×1.5 cm) of hydrated bacterial cellulose (BC) is vacuum dried down to a thickness of 2 mm in 7 minutes or after removing approximately 350 mL of solution. According to certain examples, the compressed sheet of BC has a cellulose fiber density of between 1e-3 g/mm³ to 1e-5 g/mm³. Using a laser cutter, the BC is cut into geometric-shaped layers that allow for full surface area coverage around the specific metal implant surface (such as a core). According to certain examples, the implant surface may be any appropriate material including, but not limited to, titanium, cobalt, stainless steel, and alloys of such metals. According to certain examples, the surface may or may not be porous. According to certain other examples, the geometric shapes may be radial or flower-like shapes or a string/ribbon shape.

After forming the layers of BC around the implant surface, the implant is placed in batting and vacuum sealed until the BC petals or legs adhere to one another and the metal implant surface. While the implant sits in the vacuum sealed bag, a 75 g 40% polyvinyl alcohol (PVA) solution (30 g of PVA to 45 g of H₂O) is made in the Teflon infiltration chamber. The implant is then removed from the vacuum sealed bag and smoothed to remove any wrinkling from the vacuuming process. The implant is immersed into the PVA solution within an infiltration chamber stem up, for an appropriate time and temperature range, such as >12 hours and >100° C. The infiltration chamber may be formed of a non-reactive material, such as PTFE or an appropriate polymeric material (e.g., Teflon). The implant may be annealed at an appropriate time and temperature range (e.g., >80° C. for >12 hours). After removing the implant from the infiltration chamber, excess PVA is removed from the implant by hand and the implant is compressed into its mold, for example via vacuum, pneumatic, or mechanical compression. Using a wrench and screwdriver, the mold is tightly shut. The mold is placed into the pressure chamber, pressurized, and left to cool in the freezer for 1 hour. Once the implant is pulled out of the mold, excess PVA is trimmed off the implant using a scalpel. The implant is then set into a laboratory oven at 90° C. for 24 hours to anneal. Post annealing, the excess PVA ridge left on the implant as well as any additional PVA or BC near the stem of the implant is trimmed off, e.g., with a lathe or other trimming mechanism.

In one example process the method may include pre-vacuuming BC sheets down to a thickness of 2 mm and using a cushioning material (e.g., ‘batting’) to prevent wrinkles. Although specific examples of how steps or stages of a method for preparing an implant are described, the method of performing each step may be varied.

In some examples the sheet(s) of BC material to be used may be dried in any appropriate manner, prior to cutting and shaping to the implant surface(s). For example, a vacuum oven may be used to reduce the water content of the BC material (e.g., “slabs”). To successfully form BC around a mold, fully hydrated BC slabs may remain un-compressed, as greater thickness may result in uneven pressure towards the top portion of the mold. As such, the example process described herein may involve compressing (i.e. vacuum compressing) the BC sheets down to a thickness of 2 mm prior to forming the implant. As previously mentioned, compression may also include other means of compression such as pneumatic compression or mechanical compression. Additionally, the compression or vacuuming process in the example process described herein may involve the use of batting to provide additional cushion to prevent the suction forces from severely wrinkling the BC around the implant.

To create the BC layers used during the forming stage of the procedure layers, hydrated sheets of BC (20 cm×10 cm×1.5 cm) may be vacuumed down to 2 mm in thickness/density from an original thickness/density of roughly 15 mm. At the original thickness/density, the BC sheet may be unable to successfully mold around the entire metal surface; thus, it may flatten to a thinner thickness/density, ideally within the range of 1.5 mm to 2.5 mm, while remaining hydrated, to be formed around the implant surface. An appropriate thickness/density may be within the range of 0.5-8 mm, corresponding to a cellulose fiber density of 1E-3 g/mm³ to 1E-5 g/mm³. Vacuum sealing produces a consistent thickness of the BC reinforced hydrogel coating of the BC layers across the implant and a uniform surface.

FIG. 1B shows an exemplary method 110 of forming an implant having a bacterial cellulose-hydrogel as described herein. In some cases the implant may be an implant for a femoral condyle, but these methods are not limited to femoral condyle implants. In the example shown in FIG. 1B, one or more sheets of freeze-dried bacterial cellulose (BC) is cut into a desired geometric shape or shapes 111, e.g., using a laser cutter. These shapes may form layers that may be placed around the implant core's head so that the entire surface area of the head is covered 112, and excess BC is extruding partially down the stem of the core. In FIG. 1B, the extruding portions of the sheets/layers of BC are secured down around the stem of the core 113 with an appropriate securement (e.g., clamp, band, tie, etc.) that prevents movement of the BC during the subsequent manufacturing steps. After forming these layers, the implant may be placed in a water bath to rehydrate the BC. The implant may then be removed from the water bath and placed into an infiltration chamber containing a hydrogel-forming solution, e.g., a 40% polyvinyl alcohol (PVA) solution. The infiltration chamber and implant may then be allowed to infiltrate into the BC for a desired period of time and at a preferred temperature; in FIGS. 1B, the implant with the BC is placed into an oven at 120° C. for 24 hours 114. Once the infiltrating is complete, excess PVA may be removed (e.g., by hand or via a machine), and the implant may be compressed into a mold. In some cases the implant may be chilled/frozen to assist in shaping the BC-hydrogel. For example, the mold may be left to cool in a freezer for 1 hour 115. After this, the implant may be removed from the mold and excess PVA is trimmed off if necessary. The implant may then be annealed 116. For example, the implant may be placed into a laboratory oven at 90° C. for 24 hours to anneal. After annealing, a permanent backplate 117 may then be secured (e.g., crimped) onto the undercut region over the stem of the implant 116. In some examples, exemplary process 110 for forming an implant having a BC-hydrogel secured thereto may include one or more (e.g., 2, 3, 4, 5, 6, etc.) layers of pre-cut (e.g., floral-cut) BC and a titanium implant core. The dimensions of the implant provide suitable parameters for insertion into the cortical bone of the femoral condyle with minimal penetration of the head of the implant into the trabecular bone. This results in a dense platform for the implant weight-bearing surface with minimal synovial fluid integration to reduce bone resorption.

### EXAMPLE PROCEDURE

### Laser-Cutting Bacterial Cellulose (BC)

In one example, BC sheets that meet predetermined specifications may be used during the forming stage. The characteristics of the BC may allow it to be formed into a composite hydrogel that simulates cartilage and provides sufficient tensile strength and biocompatibility for insertion into the knee. Similar to the collagen fibers in natural cartilage, the cellulose fibers in the BC can be hydrated to become composed mainly of water, making it both strong and flexible.

In some examples, the implant may include multiple equal laser-cut BC layers that may include multiple arms or petals (e.g., having a floral shape, as described).

FIG. 1C shows an exemplary pattern for a layer of laser-cut floral shaped BC. To create the curvature of the BC legs, the latitudinal circumference of the BC and PVA layer was measured at respective points along the circular section of the implant head in order to calculate the shape that is suitable to cover the head of the implant core. This shape allows the BC to cover the entire articulating surface of the implant while being clamped in place.

To form the implant, layers of BC may be oriented to cover the entire titanium surface head of the implant core 112. FIGS. 1D-1E show an example of a BC forming fixture having a BC layer without (FIG. 1D) and with (FIG. 1E) the head of an implant core, respectively. As shown here, a single layer of BC 120 is shown for simplicity, but in some examples, multiple layers may be used simultaneously in manufacturing. To aid in this process, the BC layers are laid on top of one another with no overlap and then placed concurrently onto a forming fixture 121. Next, the implant core 122 is placed face-down onto the BC layer(s) 120 and pressed into the forming fixture 121 so that it is held in place. Using forceps, the legs 123 of the BC 120 are manually wrapped around the head of the core 122.

FIG. 1F shows an example of a BC forming process and fixture 119. A securement (e.g., clamp 124) is placed around the stem of the core 122 in a manner that allows it to secure the extruding legs 123 of the BC 120 (for example clamping the legs of the BC onto the stem of the implant 113), which holds the BC 120 in place while inserted face-down into the forming fixture 121. In some examples, an aluminum backplate 125 is secured against the underside of the implant core 122 (around the stem of the core 122) before placing the clamp 124.

FIG. 1G shows an example of an infiltration process 126 for infiltrating the BC with hydrogel. Before infiltration, the BC 120 may be hydrated. To accomplish this, the implant 122 having the BC 120 on it is placed into a water bath (for example at 80° C. for 2 hours) 127. While hydrating, the infiltration chamber may be set up. In some examples, a 40% PVA solution may be used for infiltration within the infiltration chamber, which the implant is placed into 128. The PVA consists of chains of repeating molecules that form a gel. When infiltrated into the BC, the PVA gives the BC the ability to return to its original shape after being stretched.

Once the implant is finished hydrating, it is immediately placed stem up into a cup with the PVA solution. This cup is placed into a metal infiltration chamber that is sealed tightly and then put into a laboratory oven at 120° C. for 24 hours 129.

FIGS. 1H-1I show examples of an implant post-hydration. FIG. 1J shows an example of an implant before molding. The implant may be molded with a customized mold with various parts and features that allow for a smooth surface to form. When the implant is finished infiltrating, it is removed from the infiltration chamber, and visibly excess PVA 130 is removed by hand with a scalpel. The implant may then be gently compressed into the mold to ensure that no BC is damaged during the process. FIGS. 1K-1L show examples of an implant after molding.

Once the implant is in the mold, the mold is pressed shut. The mold is then placed in a freezer to cool for an hour.

FIG. 1M is an example of a disassembled two-part mold fixture. FIG. 1N is an example of an assembled two-part mold fixture. FIG. 1O is an example of a molding process 131. As shown here, an implant with BC such as those shown in FIGS. 1I-1L may be placed into a mold fixture such as the two-part mold fixture from FIGS. 1M-1N for molding.

FIGS. 1P-1R show examples of an annealed and crimped implant. After molding, implants are placed into a laboratory oven for 24 hours at 90° C. to anneal. After annealing, any visibly excess hydrogel is trimmed off. Once trimmed, all fixtures are removed from the implant, and a washer-shaped backplate is crimped onto the stem to mechanically secure the BC in place.

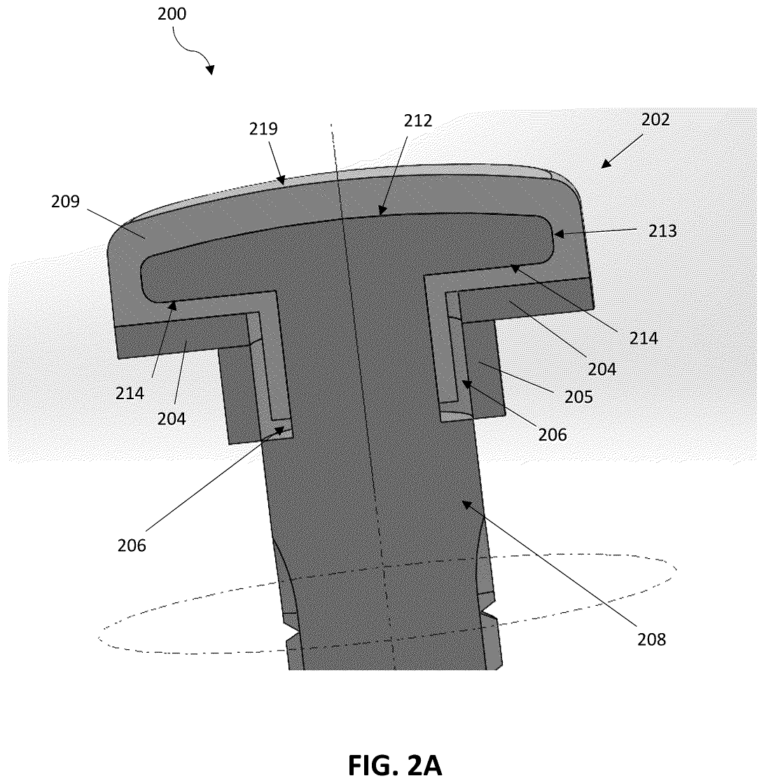

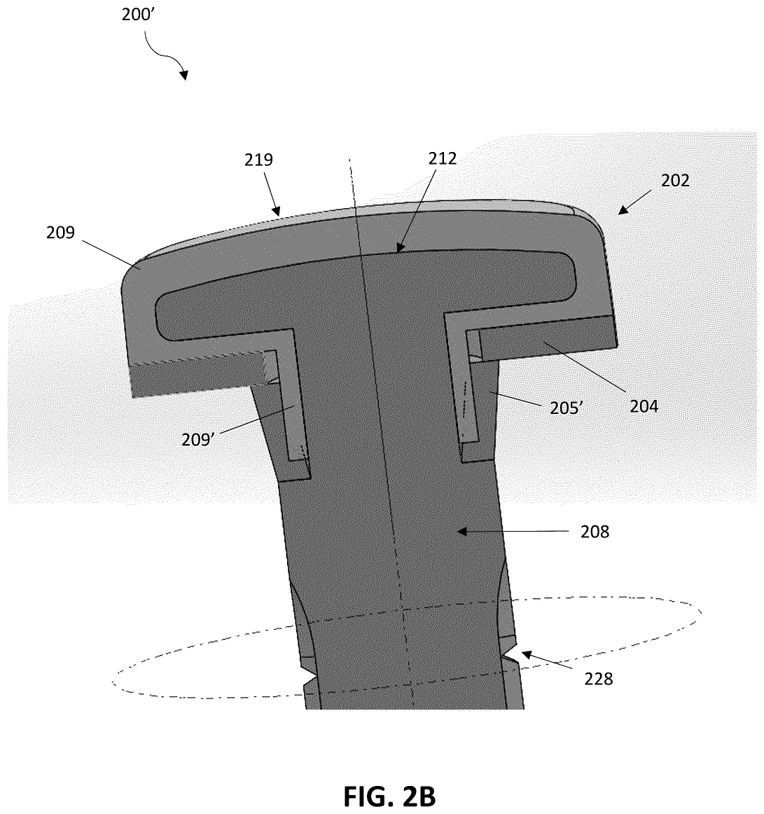

FIG. 2A shows an example of an implant 200 include a hydrogel impregnated bacterial cellulose (BC) 209. As shown here, the implant includes a head portion 202 with an upper head surface 212, a side region 213, and an undercut region 214. According to certain examples, the head of the implant 200 is covered with BC impregnated with hydrogel 209, including each of the upper surface 212, side(s) 213 and undercut region 214. The hydrogel-impregnated BC 209 is wrapped around an undercut region 214. The BC-hydrogel (e.g., BC-PVA) may be secured both by being formed into the undercut region and, in some examples, may be secured by a plate 204 (shown as a washer-like ring structured 204 in FIG. 2A) that may be secured to the undercut region 214 by crimping a ring 205 extending (e.g., perpendicular to the plate 204) over the shaft 208 of the implant. The crimping ring 205 may be crimped by applying radially-inward pressure against the crimping ring 205; in some example the implant may include a recessed region 206 of the shaft 208 to receive the crimp, as shown in FIG. 2A. The ring to be crimped may be referred to as a crimping ring or simply a crimp.

In some examples, a mold may be applied to the hydrogel-impregnated BC 209 for a smooth external surface, such as the upper surface of the implant 219. During a molding process, excess or perimeter hydrogel-impregnated BC may be removed. In FIGS. 2A-2B a portion 209′ of the hydrogel-impregnated BC 209 extends down the shaft 208 of the implant and is crimped behind the crimping ring. In some examples the crimping ring does not directly contact the BC-hydrogel material, but instead secures the plate 204. The implant head 202 may generally be coupled to one or more posts (e.g., a singe post 208 is shown in FIGS. 2A-2B), that may be configured for implantation and structural stability. In some examples the post 208 may have a plurality of engaging edges 228 along its length, configured to engage bone.

The height of the implant head may be adjusted in order to sit snugly (or slightly proud) of a matching recess within the bone into which it is to be implanted. According to some examples, head of implant 202, including the hydrogel thickness 209, may be short, for example about 2 mm instead of about 6 mm to configure implant 200 to sit flush in the cortical bone layer prevent penetration or subsidence of implant 200 beyond the cortical bone layer.

In any of these apparatuses all or a portion of the implant may include one or more coatings, such as a hydroxyapatite coating, to enhance biocompatibility and/or ingrowth, e.g., into the one or more posts (e.g., shafts) For example the implant (including in some examples, at least a portion of the BC-hydrogel) may be at least partially coated with a liquid hydroxyapatite (HA) coating. In any of these examples, either or both of a surface of the hydrogel and/or the surface of the implant may be coated with hydroxyapatite. Thus, any of these methods may include coating one or both of a surface of the hydrogel and/or the surface of the implant with hydroxyapatite.

FIG. 2B shows the implant 200′ of FIG. 2A after formation of a crimp to retain the hydrogel-impregnated BC 209. In some examples, the hydrogel-impregnated BC 209 may be secured directed in the recessed region 206 by the crimp 205. Alternatively, in some examples, the BC (and therefore BC-hydrogel) is limited to the undercut region, e.g., by trimming, etc.

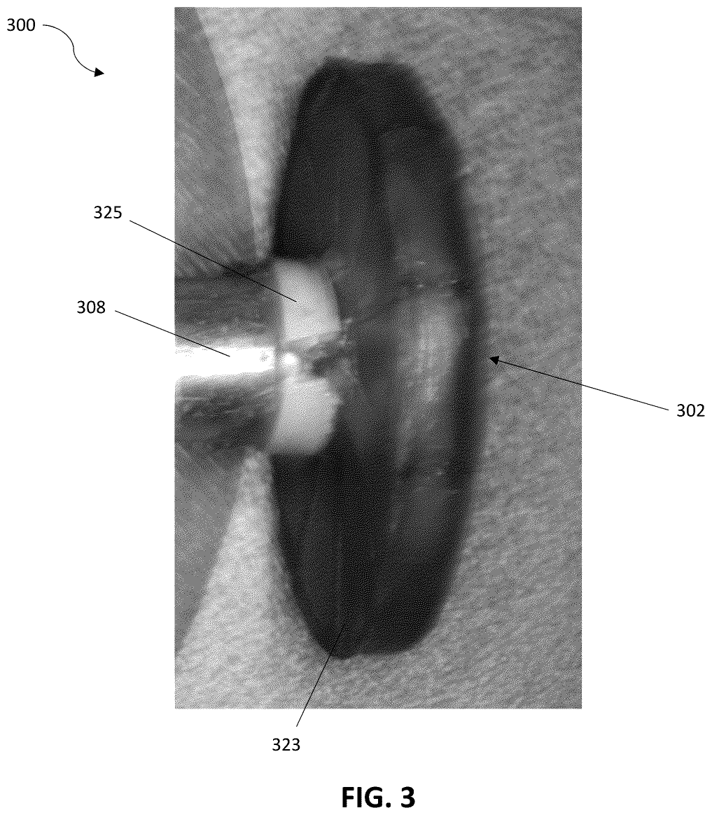

In some examples, the BC may be applied to the implant (e.g., before impregnating with hydrogel) and temporarily secured to the implant, e.g., by a temporary securement, such as a ring (as shown in FIG. 3, described in greater detail below). The BC may be applied over the undercut region prior to impregnation/infusion with the hydrogel, and may be trimmed. In some cases the BC may be pre-cut, e.g., into a petal-like pattern, so that it fits over the outer surface 212 of the head region 203 as a single piece, but may be trimmed so that it either does not overlap of partially overlaps over the outer edge(s) 213 and either overlaps or does no overlap over the undercut region 214 and/or the (optional) region extending down the post 208.

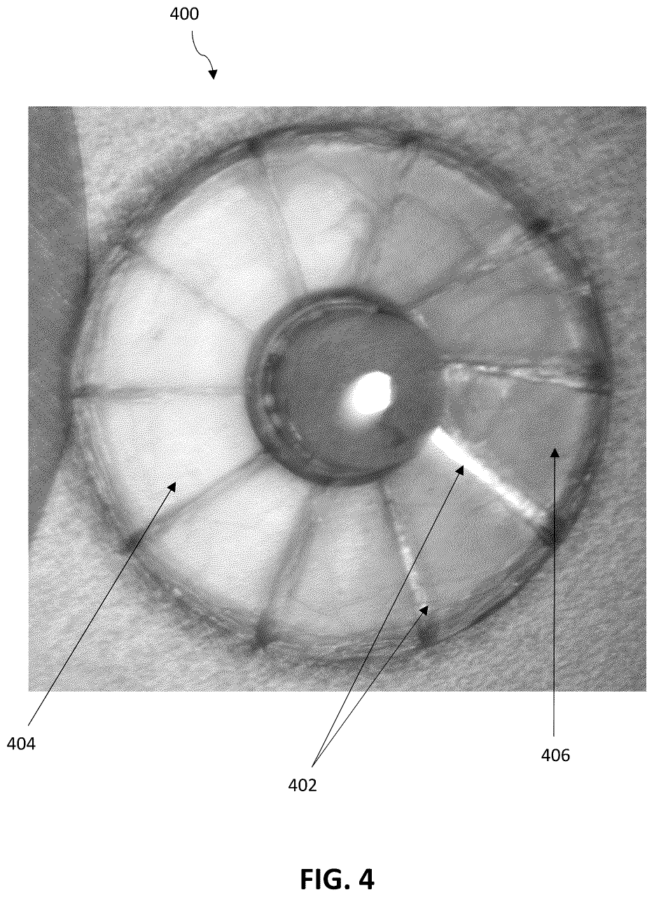

Thus, in any of these apparatuses the BC may be cut into a pattern to control the surface smoothness, overlap and/or thickness. In some examples the BC is formed into a pattern that does not overlap with itself, for example by having a pre-cut shape such as a petal-like or star shape. In some examples it may be desirable to have the BC, and therefore the hydrogel-impregnated BC 204, overlap or wrap around itself when covering the implant surfaces. The pattern of the BC on the implant may be selected or formed so that the impregnation/infusion of the hydrogel is optimized. For example the pattern of the BC on the undercut region may be formed so that channels (e.g., between adjacent regions of BC) are present, so that the hydrogel may penetrates into the BC on the undercut region, even where a temporary securement is used to hold the undercut region in place, as will be shown and described in reference to FIG. 4, below.

In FIGS. 2A-2B, the crimping ring 205, 205′ may be held in a recessed region of the neck of the implant 200, below undercut region 214. According to certain examples, the crimp may be tapered, or smaller at the bottom away from the undercut region 214 as compared to closer to undercut region 214.

In general, during formation of the BC-hydrogel covering of the head of the implant, the sheet(s) of BC may be secured to the implant head by a temporary securement to allow manipulation of the BC and/or impregnation/infusion with the hydrogel. For example, a temporary securement may be removably affixed to the shaft and/or undercut portion of the implant. To hold the BC onto the implant during fabrication, a temporary securement may be attached over the undercut region and/or to the base of the post. For example, in some cases the sheet of cut BC (e.g., cut into a star-shaped or petal-like pattern in some cases) may be applied over the head of the implant, and temporarily secured in place with a temporary securement around the base of the post; in some cases the temporary securement may include a plate that is held against the undercut region.

FIG. 3 shows a side view of an example of an implant 300 with a temporary securement including a plate 323 and a collar 325. The temporary securement may be retained while impregnating the BC and/or curing the BC-PVA. The temporary retainment may be swapped out for a retaining plate and/or crimp. For example, after annealing, a retaining plate may be applied to the undercut/underside region of the head of the implant 302, against the hydrogel impregnated BC. In some examples, a retaining plate may be coupled against the undercut region 214, as shown in FIG. 2A, to secure the hydrogel-impregnated BC 204 between the undercut region 214 and the retaining plate 310. In other examples, other retainer may be used, such as a spring (e.g., Teflon spring) or wire wrapping, to help secure the hydrogel-impregnated BC 204 to the undercut region 214, to conceal the hydrogel-impregnated BC 209 and/or to minimize direct contact between the hydrogel-impregnated BC and the bone cavity at the site of implantation.

In some case a temporary retainment is not used, and the method and apparatus may instead just use the retaining plate and/or crimp.

FIG. 4 shows an example of a bottom view of an implant with channels 402 between regions of BC that may assist in penetration of the hydrogel (e.g., polyvinyl alcohol, PVA). In the example shown in FIG. 4, the PVA has been allowed to penetrate into the BC. On the right side of the implant (undercut region) gaps or channels 402 are formed which may allow the hydrogel (e.g., PVA) to fully infiltrate into the BC 406 to form the hydrogel-impregnated BC, and the resulting composite is shown having a slightly darker color on the right side of FIG. 4. Hydration of PVA may cause filling of the channels 402 as well. On the left side of the implant shown in FIG. 4, channels were not formed (e.g., the BC overlapped or abutted) and the PVA does not fully infiltrate the hydrogel-impregnated BC, resulting in the whiter color shown.

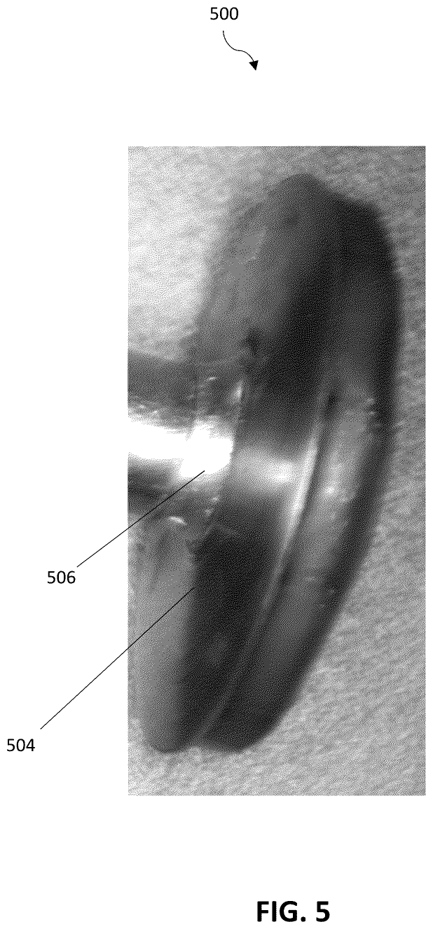

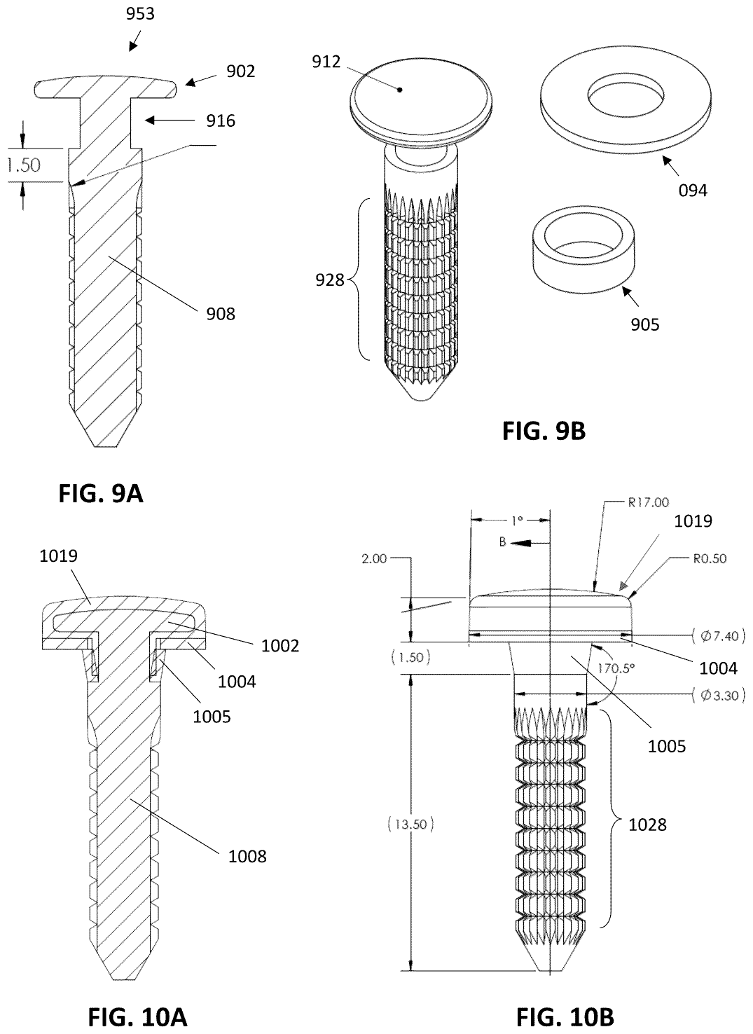

FIG. 5 is a side view of an implant with a metal backplate 504 and crimp 506. As shown here, the metal plate 504 may be configured to permanently secure the BC-hydrogel, for example after annealing. A crimp 506 may hold metal backplate 504 in place. FIGS. 9A-9B illustrate an example of components of an implant similar to that shown in FIGS. 2A-2B, 3, 4, 5 and 6. FIGS. 9A-9B illustrate an example of components of an implant without the BC-hydrogel attached. FIG. 9A shows an example of a metal core 943 of an implant such as the implant shown in FIGS. 10A-10B. The section shows a head region 902, and a recessed region 916 on the neck of the post 908 in which the crimp 905 may reside. The post 908 of the metal core may form the post of the implant, and may include one or more engaging edges 928 circumferentially and along the length of the post. The exploded view shown in FIG. 9B includes the crimp 905 as well as the plate 904 (shown configured as a washer-like structure).

FIGS. 10A-10B illustrate an implant including the metal core, crimp 1005 and retaining plate 1004 similar to those shown in FIGS. 9A-9B, as well as a BC-hydrogel 1019 extending over the top, side(s) and undercut region of the head 1002 of the implant core. The shaft portion 1008 also includes a plurality of engaging edges 1028. In any of these examples the shaft of the implant may be non-porous, but may be configured as rough, avoiding channels for flow of fluid, while providing a rough surface to enhance bone growth.

In general, the use of the retaining plate may be particularly useful to increase the shear strength of the BC-hydrogel when implanted.

The dimensions shown in FIGS. 9A-9B and 10A-10B are shown in mm, and are not intended to be limiting, but merely illustrate one example. Actual dimensions may be difference (e.g., may vary by +/−5%, 10%, 25%, 50%, 75%, 100% or more).

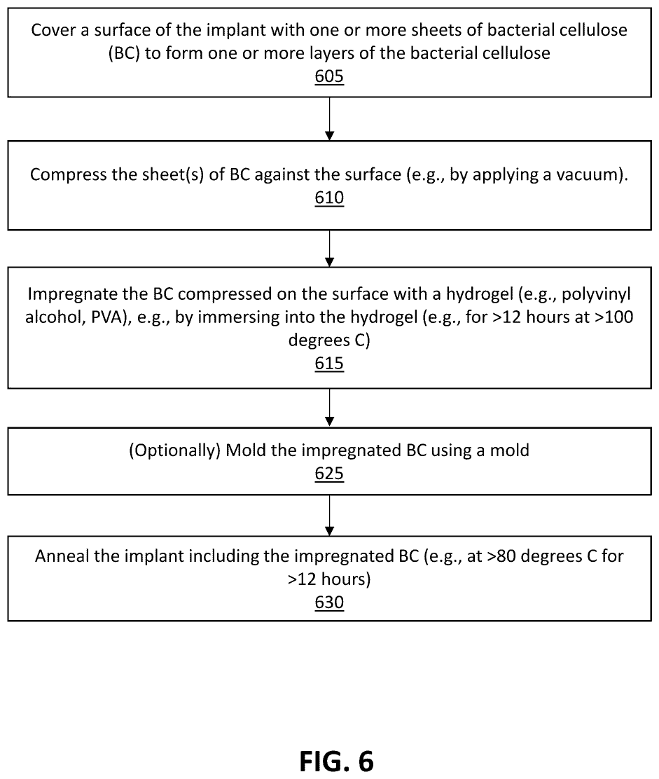

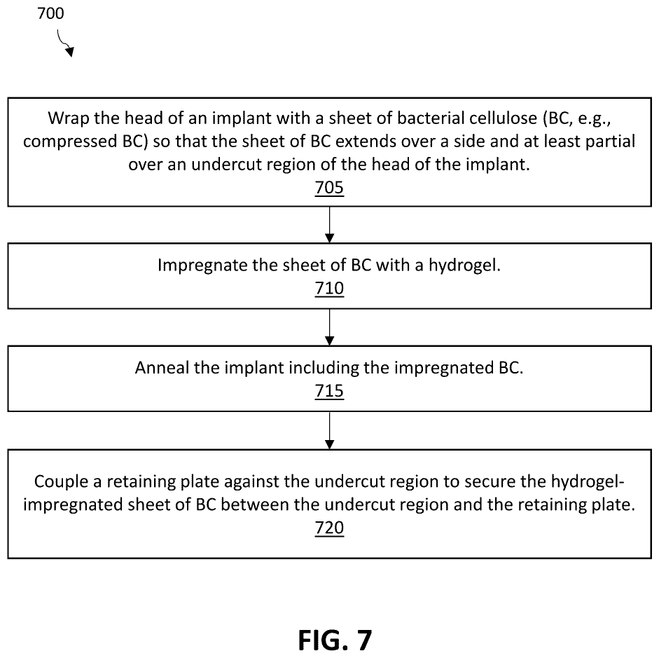

Returning now to FIGS. 6-8, these examples illustrate method of making and using the implants described herein. For example, FIGS. 6-7 schematically illustrate methods of forming an implant as described herein.

FIG. 6 shows a chart illustrating a method 600 of forming an implant as described herein. For example, the surface of the implant may be covered with a sheet (in some examples, a compressed sheet) of bacterial cellulose (BC) to form one or more layers of the BC 605. In some examples the sheet of BC may be compressed against the surface by applying a vacuum 610. Optionally, the sheet of BC may be smoothed on the surface after compressing. The BC may be impregnated with a hydrogel, such as, but not limited to, polyvinyl alcohol (PVA), e.g., by immersing it into the PVA for a desired length of time, e.g., >12 hours, at a target temperature (e.g., >100 degrees C.) 615. The implant, including the impregnated BC may then be annealed 630. Optionally, the impregnated BC may first be molded, e.g., using a mold 625. Finally, the implant with the hydrogel-impregnated BC may be annealed 630 (e.g., at greater than 80 degrees C. for more than 12 hours). Alternatively or additionally, FIG. 7 schematically illustrates a method 700 of forming an implant as described herein. An implant frame (e.g., implant without BC or hydrogel) having a head portion may be covered (e.g., “wrapped”) with a sheet of bacterial cellulose (BC) so that the sheet of BC extends over a side and over an undercut region of the head of the implant. The BC may be impregnated with a hydrogel 710. This may be followed by annealing the implant including the impregnated BC 715. Finally, in some examples a retainer (e.g., a retaining plate, wire, etc.) may be coupled against the undercut region 720 to secure the hydrogel-impregnated sheet of BC between the undercut region and the retainer.

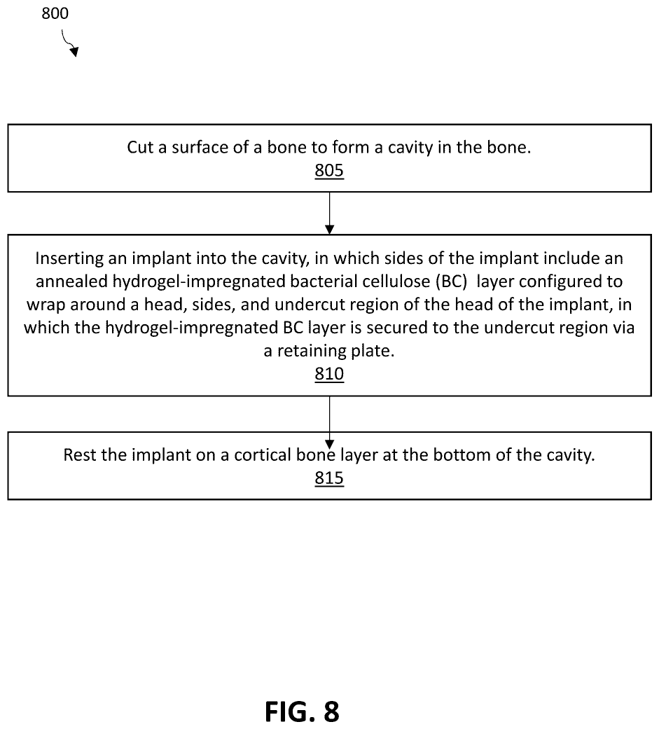

FIG. 8 schematically shows an illustrating a method of implanting an implant 800. A surface of a bone may be cut to form a cavity in the bone 805. An implant may be inserted into the cavity 810, in which sides and top of the implant may include an annealed hydrogel-impregnated bacterial cellulose (BC) layer (e.g., BC-hydrogel may cover the top, side(s), and undercut bottom regions of the head of the implant). The hydrogel-impregnated BC may be secured to the undercut region via a retainer (such as, e.g., a retaining plate, wire, etc.). The implant may be secured in the bone so that the head region is on a cortical bone layer.

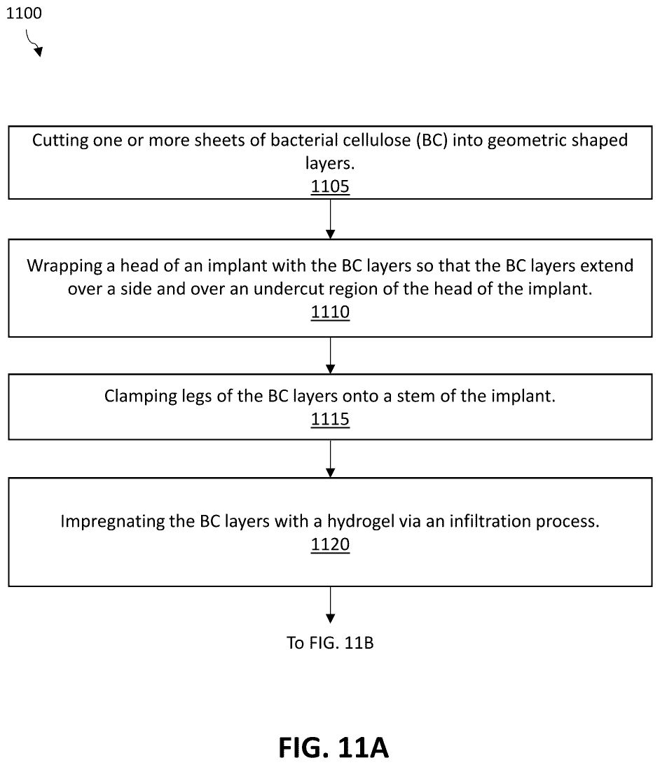

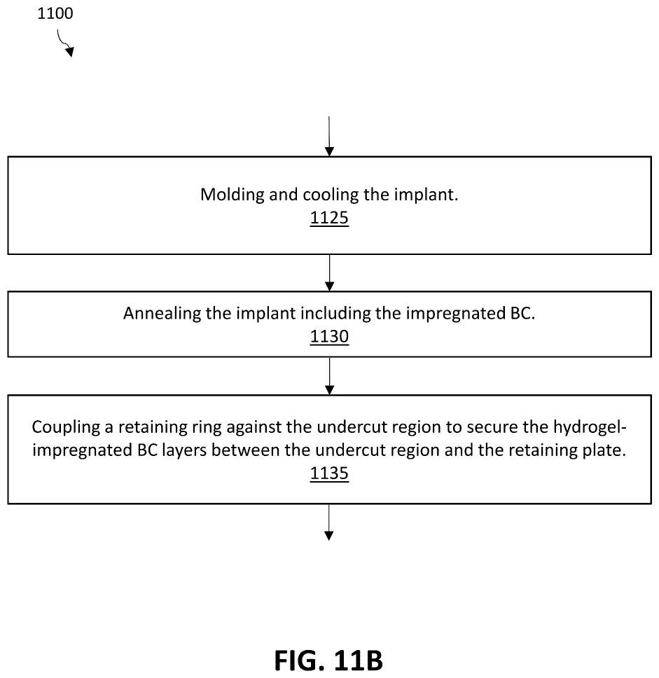

FIGS. 11A-11B schematically illustrate another method 1100 of forming an implant as described herein. Method 1100-1101 begins with cutting one or more sheets of bacterial cellulose (BC) into geometric shaped layers 1105. Next, a head of an implant may be wrapped with the BC layers so that the BC layers extend over a side and over an undercut region of the head of the implant 1110. Then, the legs of the BC layers may be clamped onto a stem of the implant 1115 and the BC layers may be impregnated with a hydrogel 1120 via an infiltration process.

The method 1100 may continue in FIG. 11B with molding and cooling the implant 1125. Next, at block 1130, the implant including the impregnated BC is annealed. Finally, at block 1135, method 1100-1101 concludes with coupling a retaining ring against the undercut region to secure the hydrogel-impregnated BC layers between the undercut region and the retaining plate.

In some examples of method 1100-1101, wrapping a head of an implant with the layers of BC further includes placing the BC layers on top of one another; concurrently placing the BC layers onto a forming fixture; placing the implant face-down onto the BC layers; and pressing the implant into the forming fixture. In some examples of method, securing the legs of the BC layers onto the stem of the implant includes: wrapping the legs of the BC layers around the head of the implant via forceps; and placing a clamp around the stem of the implant, in which the clamp clamps the legs of the BC layers. In some examples, impregnating the BC layers with a hydrogel via an infiltration process includes: hydrating the implant in a water bath at 80° C. for 2 hours; placing the implant in a 40% PVA solution and in an infiltration chamber; and infiltrating the implant at 120° C. for 24 hours.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of forming an implant, the method comprising:
wrapping a head of an implant with a sheet of bacterial cellulose (BC) so that the sheet of BC extends over a side and over an undercut region of the head of the implant;
impregnating the sheet of BC with a hydrogel;
annealing the implant including the impregnated BC; and
coupling a retaining plate against the undercut region to secure the hydrogel-impregnated sheet of BC between the undercut region and the retaining plate.

2. The method of claim 1, further comprising molding the sheet of BC under pressure after impregnating the sheet BC in order to contour the sheet of BC.

3. The method of claim 1, further comprising temporarily securing the portion of the sheet of BC against the undercut region before impregnating the BC with the hydrogel.

4. The method of claim 3, wherein temporarily securing comprises holding the sheet of BC against the undercut region with a ring that is removably secured to a post extending from the head of the implant.

5. The method of claim 1, wherein the retaining plate provides a buffering interface between the hydrogel of the hydrogel-impregnated sheet of BC and a surface of a bone configured to receive the implant, wherein coupling the retaining plate comprises coupling a retaining ring to secure the retaining plate against the undercut region, wherein the retaining ring secures the sheet of BC against the head of the implant.

6. The method of claim 5, wherein coupling the retaining ring comprises crimping the retaining ring against a post extending from the head of the implant and through the retaining plate.

7. The method of claim 1, wherein wrapping comprises leaving a gap between edges of the sheet in the undercut region.

8. The method of claim 1, further comprising cutting the sheet of BC prior to wrapping to minimize overlap of edges of the sheet on the undercut region.

9. The method of claim 8, wherein cutting comprises form a petal-shaped pattern.

10. The method of claim 1, wherein the hydrogel comprises a polyvinyl alcohol (PVA) hydrogel.

11. The method of claim 1, further comprising coating one or both of a surface of the hydrogel and/or the surface of the implant with hydroxyapatite.

12. An orthopedic implant, the implant comprising:
a head having an upper surface, an undercut region, and a side region extending between the upper surface and the undercut region;
a bacterial cellulose (BC) layer impregnated with a hydrogel covering the upper surface, the side region and at least partially over the undercut region;
a retaining plate secured against the undercut region so that the BC layer impregnated with the hydrogel is clamped between the retaining plate and a surface of the undercut region; and
a post extending from the head and through the retaining plate.

13. The implant of claim 12, wherein a portion of the BC sheet impregnated with hydrogel on the undercut region forms a plurality of channels in the BC sheet extending radially inward from the edge region.

14. The implant of claim 12, further comprising a retaining ring secured to the post and holding the retaining plate in position, wherein the retaining plate provides a buffering interface between the hydrogel of the hydrogel-impregnated BC layer and a surface of a bone configured to receive the implant, wherein the retaining ring secures the BC sheet against the head of the implant.

15. The implant of claim 12, wherein the side region is not covered by the retaining plate.

16. The implant of claim 14, wherein the retaining ring is crimped around the post.

17. The implant of claim 14, wherein the retaining ring is crimped into a recess on the post.

18. The implant of claim 12, wherein the BC sheet impregnated with hydrogel is annealed.

19. The implant of claim 12, wherein the BC sheet impregnated with hydrogel is impregnated with a polyvinyl alcohol (PVA) hydrogel.

20. The implant of claim 12, wherein the post comprises a plurality of engaging edges along the length, configured to engage the bone.

21. A method of forming an implant, the method comprising:
cutting one or more sheets of bacterial cellulose (BC) into geometric shaped layers;
wrapping a head of an implant with the BC layers so that the BC layers extend over a side and over an undercut region of the head of the implant;
clamping legs of the BC layers onto a stem of the implant;
impregnating the BC layers with a hydrogel via an infiltration process;
molding and cooling the implant;
annealing the implant including the impregnated BC; and
coupling a retaining plate against the undercut region to secure the hydrogel-impregnated BC layers between the undercut region and the retaining plate.
